(19) United States
(12) Patent Application Publication
(10) Pub. No.: US 2020/0129073 A1
(43) Pub. Date: Apr. 30, 2020

(54) TREATMENT SYSTEM

(71) Applicant: CANON KABUSHIKI KAISHA, Tokyo (JP)

(72) Inventors: Takeshi Suwa, Tokyo (JP); Koichi Suzuki, Kodaira-shi (JP); Ryuichi Nanaumi, Tokyo (JP); Ryuichi Otsu, Tokyo (JP)

(21) Appl. No.: 16/664,201

(22) Filed: Oct. 25, 2019

(30) Foreign Application Priority Data
Oct. 31, 2018 (JP) 2018-205556

(51) Int. Cl. A61B 5/00; A61N 5/06; A61B 18/14

(52) U.S. Cl. CPC A61B 5/0095; A61N 2005/067; A61B 18/14; A61N 5/0613

(57) ABSTRACT

A treatment system is provided and includes a treatment probe configured to perform treatment on a subject portion by irradiating the subject portion in a subject with light, an acoustic wave reception unit configured to receive an acoustic wave generated by irradiating the subject with light and to output a reception signal, an acquisition unit configured to acquire quantitative information about the subject portion based on the reception signal, and a display control unit configured to perform control to display the quantitative information about the subject portion.

### BACKGROUND

### Field

The present disclosure relates to a treatment system.

### Description of the Related Art

Conventionally, apparatuses for performing treatment of removing moles, flecks, tattoos, and hair using lasers (laser treatment apparatuses) have been studied in a medical field. Users of the conventional treatment apparatuses set irradiation parameters corresponding to types of treatment and then irradiate treatment targets with laser beams by directing irradiation probes thereto. High power pulse lasers are generally used as laser beam sources used in these treatment apparatuses. In addition, it is known that a photoacoustic wave is generated by a photoacoustic effect in a case where a pulsed laser beam is absorbed in a subject.

Regarding the conventional laser treatment apparatus, an operator visually checks a status of a treatment portion during laser irradiation (during treatment). However, there is an issue that it is difficult to visually check a status of a treatment target region inside a subject, and contents of the treatment (energy and a time length, etc.) vary depending on an operator. In this regard, Japanese Unexamined Patent Application Publication (Translation of PCT Application) No. 2011-500298 discusses a laser apparatus that performs treatment on a body tissue, more specifically, on a retina of a living eye and capable of that controlling the laser output automatically by monitoring an effect of light irradiation during the treatment.

According to the technique described in Japanese Unexamined Patent Application Publication (Translation of PCT Application) No. 2011-500298, a photoacoustic signal is constantly monitored in laser treatment for a fundus, and an irreversible tissue change caused by heat is estimated before the change actually occurs by performing feedback. Based on the estimated result, exposure parameters (amount of light (radiation power and beam diameter) and/or radiation duration) of light irradiation are controlled so as to achieve a selected tissue change (damage).

### SUMMARY OF THE INVENTION

In a case of treatment in which a greater variety of irradiation conditions are set, control of the exposure parameters (amount of light (radiation power and beam diameter) and/or radiation duration) of the light irradiation during the treatment is not sufficient. In other words, if contents of treatment (e.g., energy and time length) can be appropriately determined before the treatment, more appropriate treatment can be performed as compared to changing the exposure parameters of light irradiation during the treatment.

For example, in a case where light irradiation is applied to a dermatological field such as treatment for a macula, a fleck, a mole, a tattoo, and a hair root, it is necessary to set a greater variety of irradiation conditions such as an amount of light, a wavelength, and a pulse width for each trouble. In order to determine irradiation conditions, it is necessary to know a diameter (size) of a subject portion, a depth of the subject portion from a subject surface, a density of a specific substance included in the subject portion, and the like. The density of the specific substance included in the subject portion is, for example, a density of melanin in a fleck, a mole, or a hair root, a density of dye in a tattoo, and an amount of hemoglobin in blood. If quantitative information (indices) about an irradiation target is not obtained before the treatment, specific contents of the treatment (e.g., wavelength, energy, and time length) cannot be determined. Therefore, there is a risk that a treatment effect varies depending on an operator who performs the treatment.

Accordingly, the present disclosure is directed to a treatment system capable of presenting quantitative information about an irradiation target to an operator before treatment, so that even an inexperienced operator can perform the treatment using an appropriate light irradiation condition.

A treatment system includes a treatment probe configured to perform treatment on a subject portion by irradiating the subject portion of a subject with light, an acoustic wave reception unit configured to receive an acoustic wave generated by irradiating the subject with light and to output a reception signal, an acquisition unit configured to acquire quantitative information about the subject portion based on the reception signal, and a display control unit configured to perform control to display the quantitative information about the subject portion.

Further features will become apparent from the following description of exemplary embodiments with reference to the attached drawings.

### BRIEF DESCRIPTION OF THE DRAWINGS

FIG. 1 is a block diagram illustrating a configuration example of a treatment system according to a first exemplary embodiment.

FIGS. 2A to 2C are diagrams illustrating a principle for obtaining quantitative information about a subject portion according to the first exemplary embodiment.

FIG. 3 is a flowchart illustrating a light irradiation condition setting sequence according to the first exemplary embodiment.

FIG. 4 is a table illustrating a quantitative information acquisition table according to the first exemplary embodiment.

FIG. 5 is a table illustrating a light irradiation condition setting table according to the first exemplary embodiment.

FIG. 6 is a graph illustrating a relationship between an irradiation time and a tissue temperature leading to tissue degeneration.

FIG. 7 is a flowchart illustrating a light irradiation condition setting sequence according to the first exemplary embodiment.

### DESCRIPTION OF THE EMBODIMENTS

### (Configuration of Apparatus)

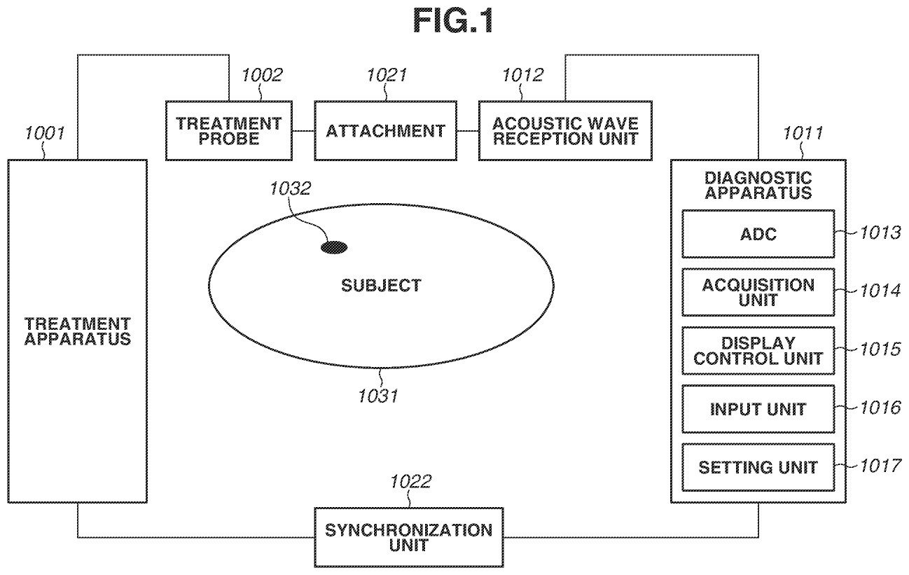

A treatment system according to a first exemplary embodiment is described with reference to FIG. 1.

FIG. 1 is a block diagram illustrating a configuration example of the treatment system according to the first exemplary embodiment. The treatment system according to the present exemplary embodiment includes at least a treatment probe 1002, an acoustic wave reception unit 1012, an acquisition unit 1014, and a setting unit 1017. The acoustic wave reception unit 1012 receives an acoustic wave generated by irradiating a subject with light to outputs a reception signal. The acquisition unit 1014 acquires quantitative information about a subject portion based on the reception signal. Thus, the treatment system can appropriately set a light irradiation condition for performing treatment on the subject portion using the obtained quantitative information. The treatment system presents the quantitative information about the subject portion to a user using a display control unit, and thus an operator (user) can appropriately set the light irradiation condition. The light irradiation condition may be automatically set without being set by the user. In any case, the light irradiation condition can be set based on the quantitative information about the subject portion, and thus the treatment for the subject portion can be performed using more appropriate light irradiation condition than a case in which information about the subject portion is visually obtained.

In FIG. 1, a subject 1031 is a skin since the present exemplary embodiment mainly targets a dermatological disease.

A subject portion 1032 includes a skin, a lesion on the skin (e.g., fleck, macula, and mole), a tattoo, and a hair root as specific examples.

According to the present exemplary embodiment, a treatment apparatus 1001 includes a laser beam source that can emit a laser beam. A specific example of the laser beam source includes a neodymium doped yttrium aluminum garnet (Nd:YAG) laser (wavelength 532 nm), a dye laser (wavelength 585 to 630 nm), and a ruby laser (wavelength 694 nm), which are used for treatment for a fleck and a macula. In addition, the laser beam source includes an alexandrite laser (wavelength 755 nm) used for a pigmentary skin disease treatment. The above-described laser beam sources employs a pulse oscillation system. In a case where the subject portion 1032 is irradiated with a pulsed laser beam and absorbs the laser beam, the subject portion 1032 thermally expands. An acoustic wave is generated from the subject portion 1032 due to the thermal expansion. In order to efficiently generate the acoustic wave, it is desirable to use a laser beam having a pulse width of several hundred nanoseconds or less. The acoustic wave herein is typically an ultrasonic wave and includes an elastic wave referred to as a sonic wave and a photoacoustic wave. A reception signal converted from an acoustic wave by the acoustic wave reception unit 1012 is also referred to as an acoustic signal. However, a description of “an ultrasonic wave” or “an acoustic wave” herein is not intended to limit a wavelength of the above-described elastic wave. An acoustic wave generated by the photoacoustic effect is referred to as a photoacoustic wave or an optical ultrasonic wave. A signal derived from a photoacoustic wave is also referred to as a photoacoustic signal. In the present specification, a photoacoustic signal is a concept including both of an analog signal and a digital signal.

The treatment probe 1002 includes an irradiation port (light irradiation portion, not illustrated) for emitting the above-described light and a spacer for securing a distance from the irradiation port to a focal position of the light. In a case where an optical system in the treatment probe 1002 is a fixed type one, and if a wavelength of the light is changed, the distance from the irradiation port to the focal position of the light is changed. Thus, it is necessary to change a length of the spacer. The treatment probe 1002 may include an optical system that changes a focal position according to a wavelength.

A diagnostic apparatus 1011 is, for example, an ultrasonic diagnostic apparatus that detects an acoustic wave (an ultrasonic wave) generated at the subject portion 1032 by irradiation of light from the treatment probe 1002 and displays the quantitative information about the subject portion 1032. A specific example of the quantitative information includes information such as a diameter (a size) of the subject portion 1032, a depth from a surface of the subject 1031 to the subject portion 1032, and a density of a specific substance included in the subject portion 1032.

The information about the depth from the surface of the subject to the subject portion is, for example, a depth from the subject surface to an edge portion nearest to the subject surface in the subject portion or a depth from the subject surface to an edge portion farthest from the subject surface in the subject portion. In addition, the information about the depth from the surface of the subject to the subject portion can be a depth from the subject surface to a center of the subject portion. If the subject portion has a circular shape, an elliptical shape, or a rectangular shape, the center is respectively the center thereof, an intersection point of a major axis and a minor axis, or an intersection point of two diagonal lines.

The information about the density of the specific substance included in the subject portion 1032 is, for example, a melanin density in a fleck, a melanin density in a mole, a melanin density in a hair root, a dye density in a tattoo, and a hemoglobin density included in blood. The information about the density may be information about a concentration and an amount in addition to a numerical value of a density.

The acoustic wave reception unit 1012 receives an acoustic wave. The acoustic wave reception unit 1012 converts the received acoustic wave into typically an analog electric signal and outputs the analog electric signal. Specifically, an ultrasonic wave transducer, which has sensitivity in a frequency band of 20 KHz of higher, can be employed. As an ultrasonic wave transducer, a piezoelectric transducer using a piezoelectric element and a capacitive transducer can be used.

An analog-to-digital converter (ADC) unit 1013 converts the analog electric signal output from the acoustic wave reception unit 1012 into a digital signal and outputs the digital signal.

The acquisition unit 1014 acquires the quantitative information about the subject portion 1032 based on the digital signal output from the ADC unit 1013. The acquisition unit 1014 can be configured by a processor, a processing circuit, a memory, and the like. The diagnostic apparatus 1011 according to the present exemplary embodiment may be configured by an amplifier that amplifies the reception signal, a memory such as a first-in first-out (FIFO) memory that stores the reception signal, and a calculation circuit such as a field-programmable gate array (FPGA) chip in addition to the units illustrated in FIG. 1. The diagnostic apparatus 1011 may be configured by a plurality of processors and calculation circuits. Each block in the diagnostic apparatus 1011 may be configured by a processing circuit having a physical entity and may be implemented as a functional block by a program module and the like.

A display control unit 1015 controls a display unit such as a liquid crystal display to display the quantitative information about the subject portion 1032 to an operator. The display control unit 1015 also displays the light irradiation condition to the operator.

An input unit 1016 receives an input from the operator. Specifically, the input unit 1016 may be a touch panel display integrated with the display unit in addition to a keyboard and a mouse.

The setting unit 1017 is used to set the light irradiation condition. Specifically, the setting unit 1017 sets at least one of an amount of light, a pulse width of light, a wavelength of light, a repetition frequency of light, and a total irradiation time of light used for performing the treatment on the subject portion.

An attachment 1021 mechanically connects the treatment probe 1002 and the acoustic wave reception unit 1012.

A synchronization unit 1022 synchronizes light irradiation from the treatment probe 1002 with an acoustic wave reception timing of the acoustic wave reception unit 1012. Specifically, a part of irradiation light from the treatment probe 1002 may be used as a reference using a photodiode (PD). In addition, an electrical signal output from a transistor-transistor logic (TTL) circuit and the like may be used as a synchronization signal. The synchronization unit 1022 may exchange setting information and the like between the treatment apparatus 1001 and the diagnostic apparatus 1011.

### (Principle for Obtaining Quantitative Information)

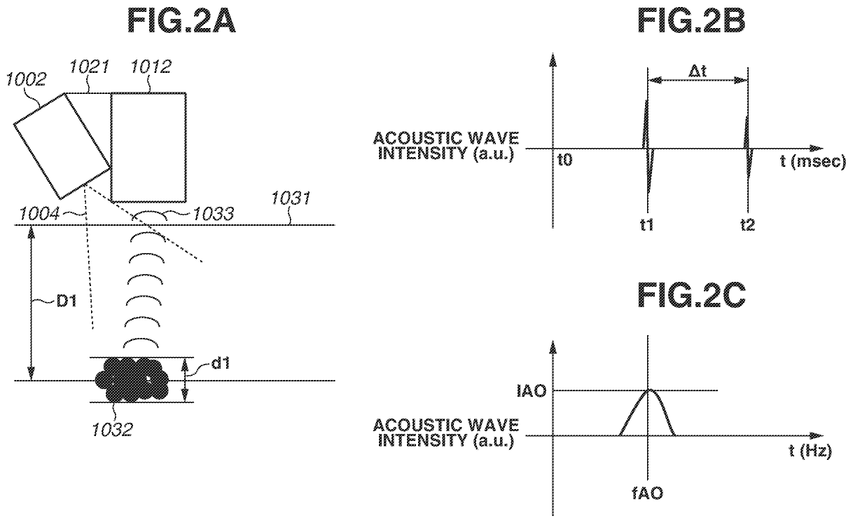

FIGS. 2A to 2C illustrate a principle for obtaining the quantitative information about the subject portion according to the first exemplary embodiment. FIG. 2A illustrates an arrangement of the subject 1031 and the treatment probe 1002 and the acoustic wave reception unit 1012 when an acoustic wave is obtained. The reference numerals used in FIG. 1 are used in FIG. 2A in the same meaning in FIG. 1.

A depth D1 is a depth of the subject portion 1032 from the surface of the subject 1031. A size d1 is a size of a cluster of the subject portion 1032. Specifically, the subject portion 1032 may include a cluster of melanin such as a fleck and a mole, a cluster of pigment of a tattoo, a hair root, and a vessel.

Irradiation light 1004 is emitted from the treatment probe 1002.

An acoustic wave (photoacoustic wave) 1033 is generated from the subject portion 1032 by irradiation of light from the treatment probe 1002.

FIG. 2B illustrates an acoustic wave signal generated at the subject portion 1032 and received by the acoustic wave reception unit 1012.

At a time t0, the treatment probe 1002 emits light. At a time t1, an acoustic wave generated at the surface of the subject 1031 is received by the acoustic wave reception unit 1012. At a time t2, an acoustic wave generated at the subject portion 1032 is received by the acoustic wave reception unit 1012. A time difference Δt is a time difference between the time t1 and the time t2. The time difference Δt corresponds to a time period from when an acoustic wave is generated at the subject portion 1032 to when the generated acoustic wave reaches the surface of the subject 1031.

In a case where a melanin density in the subject portion 1032 is higher, the subject portion 1032 absorbs more light, so that an intensity of an acoustic wave generated at the subject portion 1032 is increased.

In addition, the subject portion 1032 absorbs more light, and an internal temperature thereof easily rises. Thus, even in a case of “Selective Photothermolysis”, which uses a wavelength with high selectivity with respect to melanin and the like, there is a risk that a temperature of melanin itself rises, and surrounding normal cells are damaged by heat diffusion to the surrounding. To reduce such a damage, by setting a repetition period and a total irradiation time to irradiation conditions that do not cause damage to the surrounding normal cells in a process for setting the irradiation conditions, the damage to the normal cells surrounding the subject portion 1032 can be reduced.

FIG. 2C illustrates a signal obtained by converting a time signal in FIG. 2B to a frequency space. A center frequency fAO is a center frequency of a reception signal of a photoacoustic wave generated at the subject portion 1032. An acoustic wave intensity of the center frequency fAO mainly depends on a pulse width at a time of light irradiation and a size and concentration of the subject portion 1032. More specifically, in a case where the pulse width (nsec) at the time of light irradiation is close to a value obtained by dividing a size (mm) of the subject portion 1032 by a sound speed (mm/nsec) of the acoustic wave 1033, the acoustic wave intensity at a peak of the center frequency fAO is increased. This is because, in a case where the above-described value is small with respect to the pulse width at the time of light irradiation, the energy of the irradiation light 1004 is dispersed in a lower frequency side of the center frequency fAO and pushes the acoustic wave intensity at the peak of the center frequency fAO down. On the other hand, in a case where the above-described value is large with respect to the pulse width at the time of light irradiation, the energy of the irradiation light 1004 is dispersed in a higher frequency side of the center frequency fAO and pushes the acoustic wave intensity at the peak of the center frequency fAO down. In other words, the acoustic wave intensity at the peak of the center frequency fAO can be changed with respect to the received time signal by an influence of the pulse width of light.

Further, if the subject portion 1032 is larger, a center frequency fAO of a photoacoustic wave to be generated is shifted to the low frequency side. On the other hand, if the subject portion 1032 is smaller, the center frequency fAO of the photoacoustic wave to be generated is shifted to the high frequency side. A size of the subject portion can be estimated based on a known pulse width at the time of light irradiation by using these characteristics.

### (Operation Sequence)

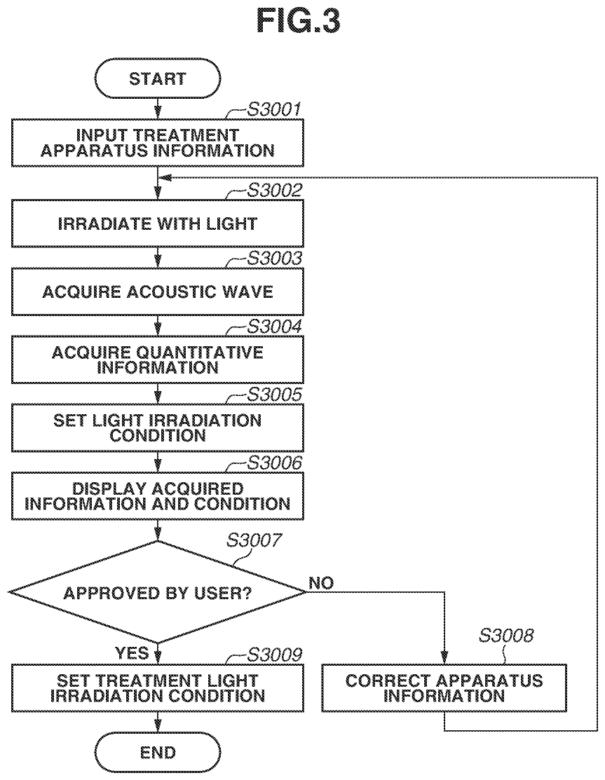

FIG. 3 is a flowchart illustrating an irradiation condition setting sequence according to the first exemplary embodiment. As described above, if a type, a region, and a size of a disease are different, irradiation conditions such as an optimum amount of light, a wavelength, a pulse width, a repetition frequency, and an irradiation time will be different.

In step S3001, treatment apparatus information (a wavelength, a pulse width, a pulse shape, and a repetition frequency of a laser set by the treatment apparatus 1001) is input to the diagnostic apparatus 1011. The input may be performed by an operator from the input unit 1016 or by communication from the treatment apparatus 1001 via the synchronization unit 1022.

In step S3002, the attachment 1021 is arranged, and light irradiation is performed. This light irradiation is not aimed at treatment, so that the light may be set to an intensity which does not have a destructive effect on the subject portion 1032 by the treatment apparatus 1001 or may be dimmed by arranged a dimming device on an optical path.

In step S3003, the acoustic wave reception unit 1012 acquires the acoustic wave 1033.

In step S3004, the acquisition unit 1014 acquires the quantitative information about the subject portion 1032 based on the acoustic wave 1033 according to a quantitative information acquisition table described below.

In step S3005, an irradiation condition of a treatment laser beam is set according to an irradiation condition setting table described below from the quantitative information about the subject portion 1032.

In step S3006, the quantitative information about the subject portion 1032 and the irradiation condition of the treatment laser beam are presented to the operator. By the presentation of the quantitative information about the subject portion 1032, even an inexperienced operator can select an optimum treatment condition from a lot of parameters.

In step S3007, the operator approves the quantitative information about the subject portion 1032 and the condition presented. If there is no problem, the operator approves the condition via the input unit 1016 (YES in step S3007), and the processing proceeds to step S3009. If there is any problem (NO in step S3007), the processing proceeds to step S3008.

In step S3008, the operator or the diagnostic apparatus 1011 corrects an input content of the treatment condition, and the sequence is repeated again from step S3001.

In step S3009, the irradiation condition is set to the treatment apparatus 1001. The irradiation condition may be set to the treatment apparatus 1001 by the operator or by the ultrasonic diagnostic apparatus 1011 using inter-device communication. If the operator sets the irradiation condition, it is desirable to confirm that the irradiation conditions coincide with each other between the treatment apparatus 1001 and the diagnostic apparatus 1011 by the inter-device communication. In this way, the optimum treatment condition can be surely reflected to irradiation. In a case where a set wavelength of the treatment apparatus 1001 is changed, it is necessary to change the optical system (light collecting condition) of the treatment probe 1002. Generally, the optical system of the treatment probe 1002 is often changed by replacing parts. However, in a case of the configuration in which the setting of the treatment apparatus 1001 is changed from the diagnostic apparatus 1011 without being changed by the operator, it is desirable to automatically change an arrangement of the optical system of the treatment probe 1002 to prevent a replacement miss of the parts by the operator. In addition, the treatment probe 1002 may be enabled to perform inter-device communication with the treatment apparatus 1001 and/or the diagnostic apparatus 1011 and be prohibited from irradiating the subject 1031 with light in a case where a corresponding wavelength of the treatment probe 1002 is different from the set wavelength of the treatment apparatus 1001. At this time, it is desirable to provide display for informing the operator that the wavelengths are different or informing the operator to replace the treatment probe 1002 with an appropriate one. It is further desirable to display a model number of an appropriate treatment probe and the like. Accordingly, the operator can easily take an appropriate action based on the display.

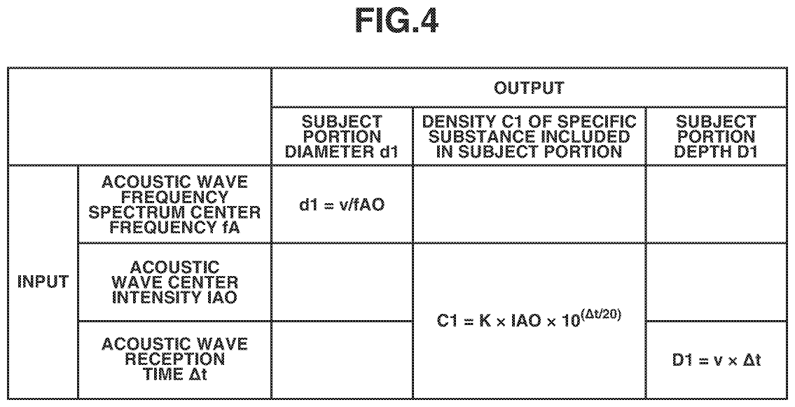

Next, the quantitative information acquisition table described in step S3004 is described in detail. FIG. 4 is an acquisition table of the quantitative information about the subject portion according to the first exemplary embodiment. The quantitative information to be output includes the diameter d1 of the subject portion, a density of the specific substance included in the subject portion or an amount C1 (herein after also referred to as density C1) in proportion thereto, and the depth D1 of the subject portion. Information to be input includes an acoustic wave frequency spectrum center frequency fA, an acoustic wave center intensity IAO, and an acoustic wave reception time Δt. Relationships between the inputs and the outputs are expressed by formulae in the table. The formulae are specifically described below.

First, the diameter d1 of the subject portion is calculated as d1=v/fAO, where v is a sound speed of the acoustic wave 1033 and is approximately 1.5 mm/μsec in a living organism.

Next, the depth D1 of the subject portion is calculated as D1=v×Δt.

Next, the density of the specific substance included in the subject portion or the amount C1 in proportion thereto is calculated as C1=K×IAO. In a case where the energy of the irradiation light 1004 is converted into acoustic wave energy, the acoustic wave center intensity IAO is increased in proportion to the density C1 of the specific substance included in the subject portion. A correction coefficient K is used for correcting an attenuation amount to an emitting end of the treatment probe 1002 with respect to an amount of light set at the treatment apparatus 1001 and further for calculating the density C1 of the specific substance included in the subject portion from the acoustic wave center intensity IAO. It is further desirable to correct an attenuation amount at the time when the acoustic wave 1033 propagates in the living organism in order to calculate the density C1 of the specific substance included in the subject portion. In addition, it is desirable to perform irradiation of light having a known pulse wave form and to use the pulse wave form subjected to deconvolution with respect to a reception signal in order to more accurately calculate the acoustic wave frequency spectrum center frequency fA and the acoustic wave center intensity IAO.

As described above, the acoustic wave frequency spectrum center frequency fA, the acoustic wave center intensity IAO, and the acoustic wave reception time Δt are calculated from the acoustic wave 1033. Further, the diameter d1 of the subject portion, the density C1 of the specific substance included in the subject portion, and the depth D1 of the subject portion, which are the quantitative information about the subject portion 1032, can be calculated.

Next, the irradiation condition setting table described in step S3005 is described in detail.

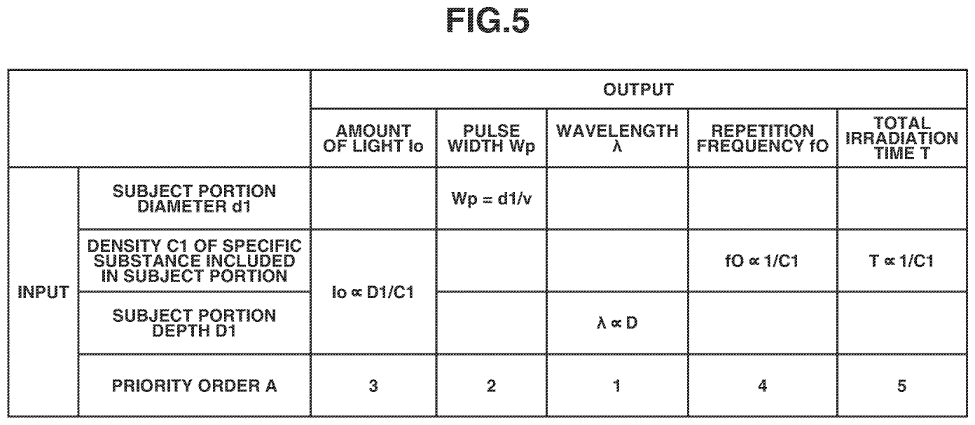

FIG. 5 illustrates the irradiation condition setting table according to the first exemplary embodiment.

Inputs in the table are the quantitative information about the subject portion 1032 obtained in step S3004 and a priority order A.

The priority order A is an order to calculate each output value. The reason why the priority order is set is described. In the general treatment apparatus 1001, settable conditions such as options of settable wavelengths and a pulse width are limited. Accordingly, the priority order A is set to perform a setting preferentially from an item with a limited condition, so that the setting of the treatment apparatus 1001 can be performed to the end without reworking. The priority order A may be presented by the diagnostic apparatus 1011 to the operator or may be set by the operator. In FIG. 5, only a proportional relationship of each output is written. This is because a conversion coefficient varies depending on the treatment apparatus 1001 to be combined with the diagnostic apparatus 1011 and is not uniquely determined. The priority order A can be used similarly regardless of a value of the conversion coefficient.

Next, the irradiation conditions, which are outputs in the irradiation condition setting table, are described.

An amount of light Io indicates the intensity of the irradiation light 1004. The amount of light Io is set to the treatment apparatus 1001. If the density C1 of the specific substance included in the subject portion 1032 becomes higher, light absorption efficiency becomes higher. Thus, a treatment effect can be obtained even with weak light. In other words, as the density of the specific substance included in the subject portion is higher, the amount of light can be reduced. In contrast, as the density is lower, the amount of light is to be increased.

Further, if the depth D1 of the subject portion 1032 becomes deeper, light is attenuated in the living organism, and the amount of light reaching the subject portion 1032 is reduced. As a result, it is necessary to set the amount of light depending on the depth D1 of the subject portion. In other words, as the depth of the subject portion is deeper, the amount of light is to be increased, and as the depth of the subject portion is shallower, the amount of light is to be reduced.

Based on the above-described matter, the amount of light Io is calculated as Io proportional to D1/C1.

A pulse width Wp indicates duration of the irradiation light 1004. In a case where a value obtained by dividing the diameter d1 of the subject portion 1032 by the sound speed v is the pulse width Wp, an acoustic wave having the acoustic wave frequency spectrum center frequency fA is generated most efficiently, and a high treatment effect can be obtained. Therefore, the pulse width Wp is calculated as Wp=d1/v.

In other words, as the diameter of the subject portion is larger, the pulse width is made larger, and as the diameter of the subject portion is smaller, the pulse width is made smaller.

The irradiation light 1004 has a wavelength λ. As the wavelength λ of the irradiation light 1004 is longer, the irradiation light can reach inside of the subject 1031. Therefore, the wavelength λ is in proportion to the depth D1 of the subject portion 1032 as a treatment target and is calculated as λ proportional to D1.

In other words, as the depth is deeper, the wavelength of light is made longer, and as the depth is shallower, the wavelength of light is made shorter.

A repetition frequency fO is a frequency of repeating irradiation of pulsed light of the irradiation light 1004. As the density C1 of the specific substance included in the subject portion 1032 is higher, the light absorption efficiency becomes higher, and the treatment effect can be obtained by a lower repetition frequency. Thus, in a case where the density C1 of the specific substance is high, the repetition frequency fO can be set lower. Therefore, the repetition frequency fO is calculated as fO proportional to 1/C1. In contrast, as the density C1 of the specific substance is lower, the higher repetition frequency is set.

A total irradiation time T is a total irradiation time. As the density C1 of the specific substance included in the subject portion 1032 is higher, the light absorption efficiency becomes higher, and the treatment effect can be obtained in a shorter total irradiation time T. Thus, in a case where the density C1 of the specific substance is high, an irradiation time can be set shorter. Therefore, the total irradiation time T is calculated as T proportional to 1/C1. In contrast, as the density C1 of the specific substance is lower, a longer total irradiation time is set.

The above-described proportionality coefficients vary depending on a combination of the treatment apparatus 1001 and the diagnostic apparatus 1011, and thus it is desirable to determine the proportionality coefficients using a phantom having quantitative information known beforehand.

Both of the repetition frequency fO and the total irradiation time T are functions including the density C1 of the specific substance included in the subject portion as a variable. If the repetition frequency fO becomes higher, a speed of a temperature rise in the subject portion 1032 and surrounding tissues during the treatment is increased. If the temperature rises too high, tissue degeneration (damage) by heat is caused in the surrounding tissues, and low temperature burn is caused. The tissue degeneration is caused in a short time at a high temperature and in a long time at a lower temperature.

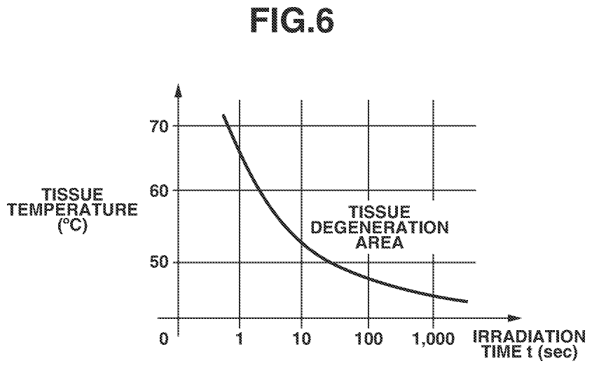

FIG. 6 illustrates a relationship between an irradiation time and a tissue temperature leading to tissue degeneration.

In FIG. 6, a horizontal axis and a vertical axis respectively indicate an irradiation time and a tissue temperature. A curve in FIG. 6 represents a tissue degeneration temperature, and the tissue degeneration starts in an upper right area of the curve.

In order not to cause excessive damage to the surrounding tissues of the subject portion 1032 during the treatment, it is desirable to set a condition based on which the surrounding tissues does not enter a tissue degeneration area.

In actual laser treatment, a situation intricately changes depending on presence or absence of a blood flow, non-uniformity of a tissue, and presence or absence of sense of cold, and thus it is desirable to set the irradiation condition in consideration of these conditions. In order to consider these conditions, it is determined whether the light irradiation condition is in the tissue degeneration area in FIG. 6 from the repetition frequency fO and the total irradiation time T. In a case where the light irradiation condition is in the tissue degeneration area, it is desirable to notify the operator of the fact and presents the irradiation condition re-set by changing the condition of the repetition frequency fO or the total irradiation time T so that the light irradiation condition is out of the tissue degeneration area to the operator.

After the quantitative information is determined from the acoustic wave obtained in step S3003, a probe group (treatment probe 1002 and/or acoustic wave reception unit 1012 and/or attachment 1021) may be temporarily removed from the subject 1031 for an input operation or the like by the operator. In a case where the probe group is set again at the subject 1031 for irradiation of the treatment laser beam, it is desirable to set the probe group at a same position as a position at which the quantitative information is determined so as to appropriately reflect the set irradiation condition to the subject portion 1032. Accordingly, the acoustic wave obtained in step S3003 is stored in the diagnostic apparatus 1011, and a second acoustic wave is obtained by executing the processing in steps S3002 and S3003 after setting the probe group again. The probe group is moved so that amplitudes of the stored acoustic wave and of the second acoustic wave approximately match with each other, and thus the probe group can be set to a desired position, i.e., the position of the subject portion 1032. At this time, in a case where it is not confirmed that the amplitudes approximately match with each other, only light irradiation is performed without irradiation of the treatment laser beam, and in a case where it is confirmed that the amplitudes approximately match with each other, irradiation of the treatment laser beam may be performed. In this way, a region except for the subject portion 1032 can be prevented from being unnecessarily irradiated with the treatment laser beam. The operator may be notified of the approximate matching of the amplitudes, and the operator who received the notification may start irradiation of the treatment laser beam. Safety can be enhanced by including confirmation by the operator. The approximate matching of the amplitudes may be determined by an appropriate algorithm in the diagnostic apparatus 1011 and may be determined by the operator watching both of waveforms of the amplitudes displayed on the diagnostic apparatus 1011. At this time, regarding a temporal position of reception signals for comparing the amplitudes, the reception signals may be compared at a time corresponding to the depth D1 of the subject portion. For example, in a case where a vascular symptom such as a port-wine stain is treated, the approximate matching of the amplitudes is confirmed with respect to not a signal from a melanin layer in a shallow portion but a signal from a blood vessel layer in a deep portion. Further, in a case where melanotic symptom is treated, it is desirable to confirm the approximate matching of the amplitudes with respect to a signal from the melanin layer, not a signal from the blood vessel layer. In this way, the position of the subject portion 1032 can be accurately detected. To confirm the approximate matching of the amplitudes, it may be determined that the amplitudes approximately match with each other in a case where the amplitudes are higher or lower than a threshold value of a reference. For example, a third acoustic wave is obtained in advance by executing the processing in steps S3002 and S3003 in a normal region except for the subject portion 1032, and a threshold value for discriminating an amplitude of the second acoustic wave and an amplitude of the third acoustic wave can be set.

Next, a light irradiation condition setting sequence for setting the priority order is described.

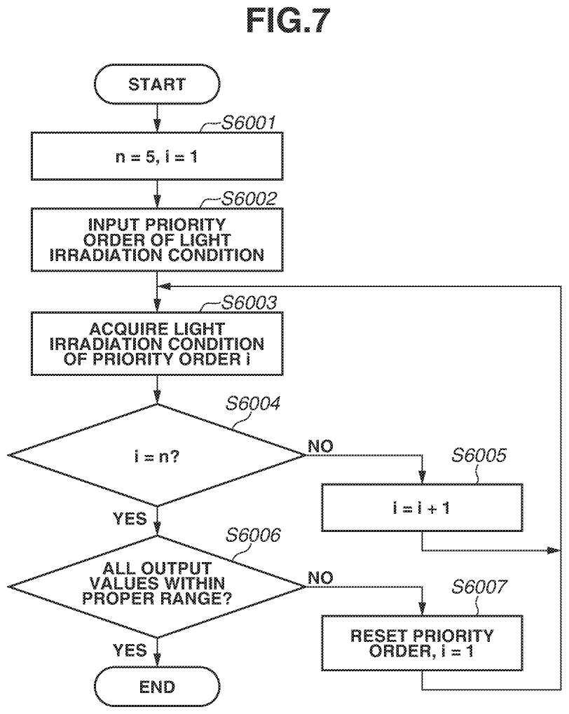

FIG. 7 is a light irradiation condition setting sequence according to the first exemplary embodiment.

In step S6001, assume that a type of the light irradiation condition to be set is n, and the priority order being set is i. In the description, assume that the number of parameters is five, i.e., n=5, and the priority order is set from the first priority (i=1).

In step S6002, the type n of the light irradiation condition is set to each priority order i.

According to the first exemplary embodiment, the priority order is higher in the order of the amount of light Io, the pulse width Wp, the wavelength λ, the repetition frequency fO, and the total irradiation time T. The order may be determined by the operator or the diagnostic apparatus 1011 in consideration of a setting range of each irradiation condition of the treatment apparatus 1001.

In step S6003, the light irradiation condition for the priority order i is acquired.

In step S6004, the priority order i is compared with the type n of the light irradiation condition. In a case of “i=n” (YES in step S6004), it is determined that all of the light irradiation conditions are set, and the processing proceeds to step S6006. In a case where “i=n” is not satisfied (NO in step S6004), the processing proceeds to step S6005.

In step S6005, a value of i is incremented, the processing is returned to step S6003, and the light irradiation condition for a next priority order i is obtained.

In step S6006, it is checked whether all of the irradiation conditions in the priority orders i=1 to n are within a proper range (e.g., within a range that can be set by the treatment apparatus). It is desirable that the operator approves a confirmation result. In a case where the operator approves the result (YES in step S6006), the irradiation condition setting is completed. In a case where the operator disapproves the result (NO in step S6006), the processing proceeds to step S6007.

In step S6007, the priority order is reset, and “i=1” is reset. Then, the processing returns to step S6003, and the light irradiation condition for the priority order i is obtained.

The treatment system according to the present exemplary embodiment can present information for determining contents of treatment (energy and a time length, etc.) to an operator before the treatment. More specifically, quantitative indices of an irradiation target such as a melanin density in a fleck, a mole, or a hair root, a dye density in a tattoo, a density of hemoglobin in the blood, and amounts in proportion to these values can be presented to the operator.

In a case where the light irradiation condition is obtained, pieces of the quantitative information about the subject portion and about a region except for the subject portion (normal region) may be presented, and the light irradiation condition may be determined based on both pieces of the quantitative information. Information that can be obtained from the region except for the subject portion includes a color of skin of the subject. Generally, light is partly absorbed on a surface of a subject because of an individual difference of a color of skin of a subject person, and an amount of light to reach the inside of the subject varies. In a case where the color of the skin is dark, it is necessary to adjust the amount of light and the irradiation time since too strong light may cause damage to surrounding skin. According to the present exemplary embodiment, both pieces of the information about the subject portion and the region except for the subject portion are presented using a photoacoustic signal, and the light irradiation condition is set based on the information, so that appropriate treatment can be performed on patients of various races with different skin color.

As described above, the configuration with which the light irradiation condition is set based on the information about the subject portion and the region except for the subject portion can present the quantitative indices of the irradiation target for determining the contents of the treatment (energy and a time length, etc.) to the operator before the treatment.

The treatment system according to the present disclosure can obtain quantitative information about a light irradiation target before treatment for a subject portion, and thus can appropriately set a light irradiation condition for the treatment for the subject portion.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2018-205556, filed Oct. 31, 2018, which is hereby incorporated by reference herein in its entirety.

What is claimed is:

1. A treatment system comprising:
a treatment probe configured to perform treatment on a subject portion by irradiating the subject portion of a subject with light;
an acoustic wave reception unit configured to receive an acoustic wave generated by irradiating the subject with light and to output a reception signal;
an acquisition unit configured to acquire quantitative information about the subject portion based on the reception signal; and
a display control unit configured to perform control to display the quantitative information about the subject portion.

2. The treatment system according to claim 1, further comprising a setting unit configured to set an irradiation condition of light for performing treatment on the subject portion based on the quantitative information about the subject portion.

3. The treatment system according to claim 1, wherein the display control unit performs control to display an irradiation condition of light for performing treatment on the subject portion.

4. The treatment system according to claim 2, wherein the irradiation condition of light is at least one of an amount of light, a pulse width of light, a wavelength of light, a repetition frequency of light, and a total irradiation time of light for performing treatment on the subject portion

5. The treatment system according to claim 1, wherein the quantitative information about the subject portion is at least one of a diameter of the subject portion, information about a depth from a surface of the subject to the subject portion, and information about a density of a specific substance included in the subject portion.

6. The treatment system according to claim 1, wherein the subject portion is one of a fleck, a mole, or a tattoo.

7. The treatment system according to claim 5, wherein the density of the specific substance included in the subject portion is at least one of a melanin density in a fleck, a melanin density in a mole, and a dye density in a tattoo.

8. The treatment system according to claim 2, wherein the setting unit sets the pulse width depending on a diameter of the subject portion.

9. The treatment system according to claim 8, wherein the setting unit increases the pulse width as the diameter of the subject portion is larger.

10. The treatment system according to claim 2, wherein the setting unit sets at least one of an amount of light and a wavelength of light corresponding to a depth from a surface of the subject to the subject portion.

11. The treatment system according to claim 10, wherein the setting unit increases the amount of light as the depth increases.

12. The treatment system according to claim 10, wherein the setting unit increases the wavelength of light as the depth increases.

13. The treatment system according to claim 2, wherein the setting unit sets at least one of an amount of light, a repetition frequency of light, and a total irradiation time of light corresponding to a density of a specific substance included in the subject portion.

14. The treatment system according to claim 13, wherein the setting unit reduces the amount of light as the density of the specific substance included in the subject portion is higher.

15. The treatment system according to claim 13, wherein the setting unit reduces the repetition frequency of light as the density of the specific substance included in the subject portion is higher.

16. The treatment system according to claim 13, wherein the setting unit reduces the total irradiation time of light as the density of the specific substance included in the subject portion is higher.

17. The treatment system according to claim 2, wherein the setting unit sets the irradiation condition of light based on information about the subject portion and a region except for the subject portion in the subject.

18. The treatment system according to claim 17, wherein the information about the region except for the subject portion is a color of a skin of the subject.
